(19) United States
(12) Patent Application Publication
(10) Pub. No.: US 2018/0055692 A1
(43) Pub. Date: Mar. 1, 2018

(54) AIM ASSISTING SYSTEMS

(71) Applicant: Diane Maddie, Port Barre, LA (US)

(72) Inventor: Diane Maddie, Port Barre, LA (US)

(21) Appl. No.: 15/254,904

(22) Filed: Sep. 1, 2016

(51) Int. Cl. A61F 9/04; F41G 11/00; A41D 20/00

(52) U.S. Cl. CPC A61F 9/045; F41G 11/00; A41D 2400/22; A41D 2600/108; A41D 2400/10; A41D 20/00

(57) ABSTRACT

An aim assisting system for assisting user's while aiming. The device includes a band and a retractable eye patch. The eye patch is designed to cover a non-dominant eye of the user thus improving accuracy and aim while shooting. A user may also use the device as an ear warmer.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. Field of the Invention

The present invention relates generally to the field of hunting equipment accessories and more specifically relates to aim assisting system entitled, “Aim Aid”.

### 2. Description of Related Art

Hunting is the practice of killing or trapping any animal, or pursuing or tracking it with the intent of doing so. Hunting wildlife or feral animals is most commonly done by humans for food, recreation, to remove predators which are dangerous to humans or domestic animals, or for trade. In the 2010s, lawful hunting is distinguished from poaching, which is the illegal killing, trapping or capture of the hunted species. The species that are hunted are referred to as game or prey and are usually mammals and birds Aiming while hunting in various conditions can be difficult. A suitable solution is desired.

Several attempts have been made to solve the above-mentioned problems such as those found in U.S. Pat. No.: 7,147,320 to Theodore Werner; and 1,340,579 to Tousey Sinclair. This art is representative of hunting equipment accessories. However, none of the above inventions and patents, taken either singly or in combination, is seen to describe the invention as claimed.

Preferably, a hunting equipment accessory should provide a means for covering a non-dominant eye and allowing a user to aim more accurately and, yet would operate reliably and be manufactured at a modest expense. Thus, a need exists for a reliable aim assisting system to avoid the above-mentioned problems.

### BRIEF SUMMARY OF THE INVENTION

In view of the foregoing disadvantages inherent in the known hunting equipment accessory art, the present invention provides a novel hunting equipment accessory. The general purpose of the present invention, which will be described subsequently in greater detail is to provide an aim assisting system.

An aim assisting system is disclosed in a preferred embodiment comprising: a body including a band having a cover having, an inner surface, and an outer surface, an insulator, an eye patch having a front surface, and a back surface. The aim assisting system comprises the body. The body comprises in functional combination the band and the eye patch. The band comprises the cover and the eye patch. The cover comprises the inner surface and the outer surface. The eye patch may comprise the front surface and the back surface. The eyepatch is integral to the band, the eyepatch affixed parallel to the band. The back surface of the eye patch is adjacent a non-aiming-eye of a user-wearer during use to facilitate ease of focus by an aiming-eye such that vision from the non-aiming-eye is blocked while shooting.

The present invention holds significant improvements and serves as an aim assisting system. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, aim assisting systems, constructed and operative according to the teachings of the present invention.

FIG. 1 shows a perspective view illustrating an aim assisting system during an ‘in-use’ condition having a band and an eye patch according to an embodiment of the present invention.

FIG. 2 is a perspective view illustrating the band comprising a cover and an insulator according to an embodiment of the present invention of FIG. 1.

FIG. 3 is a perspective view illustrating the band according to an embodiment of the present invention.

FIG. 4 is a perspective view illustrating the band according to an embodiment of the present invention.

The various embodiments of the present invention will hereinafter be described in conjunction with the appended drawings, wherein like designations denote like elements.

### DETAILED DESCRIPTION

As discussed above, embodiments of the present invention relate to a hunting equipment accessory and more particularly to an aim assisting system as used to improve aiming while shooting firearms or other hunting items.

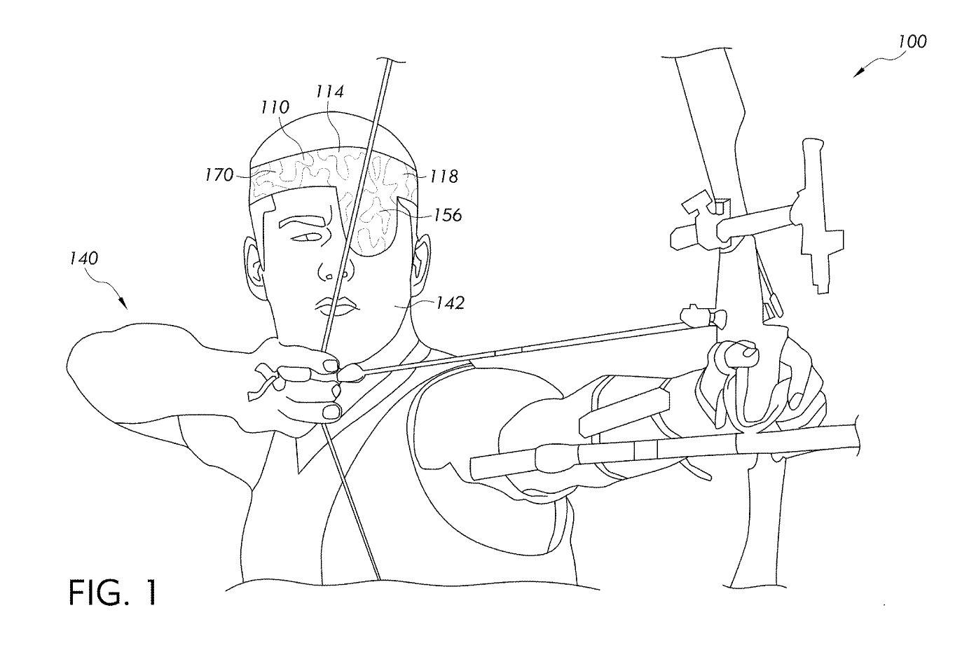

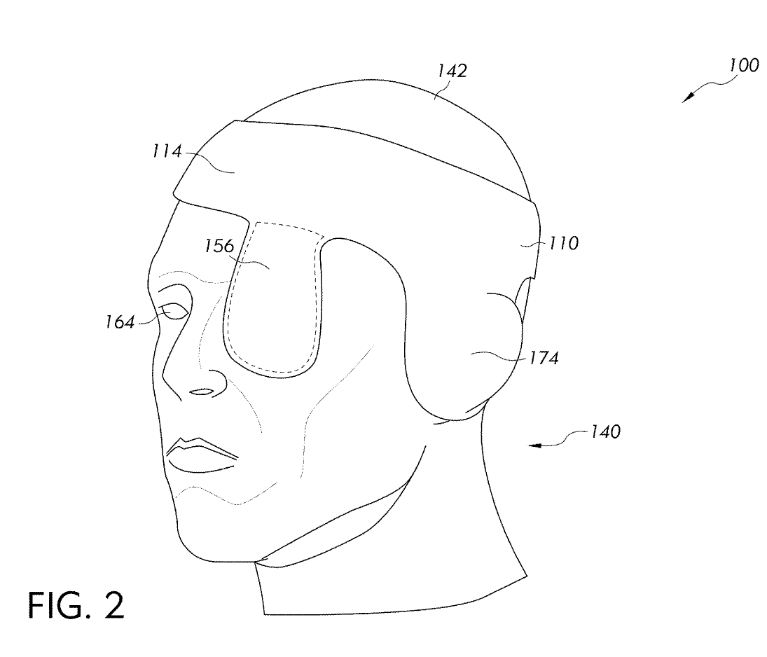

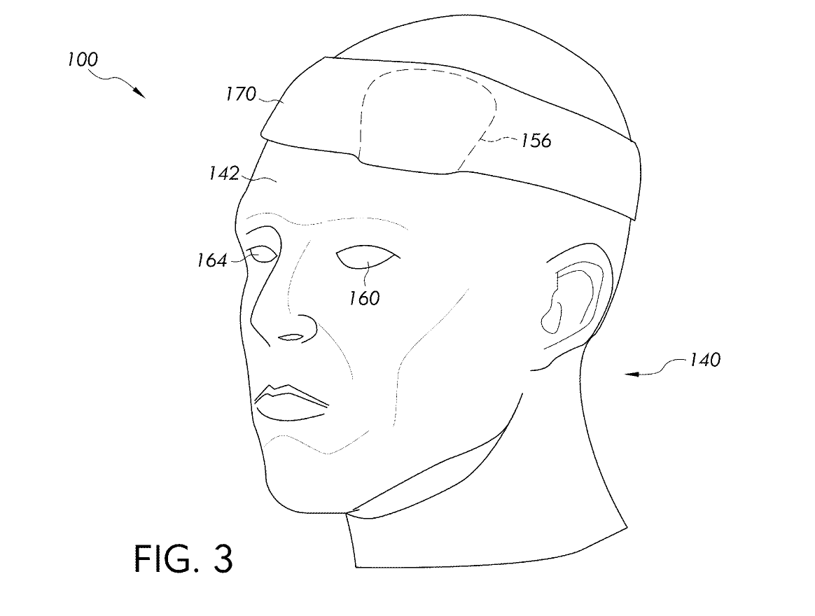

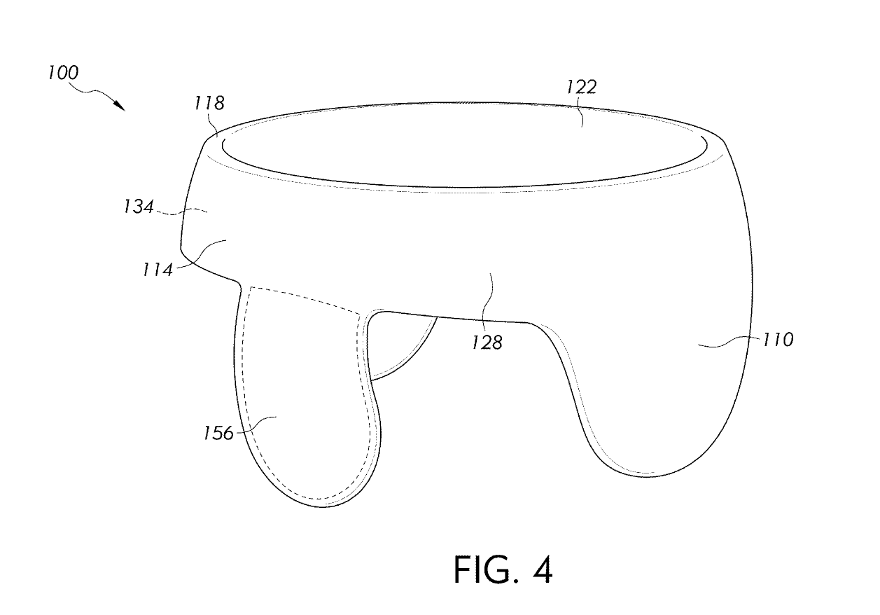

Referring to the drawings by numerals of reference there is shown in FIGS. 1-4, aim assisting system 100 according to an embodiment of the present invention. Aim assisting system 100 may comprise body 110, which may comprise band 114 having cover 118 having inner surface 122, and outer surface 128, insulator 134, eye patch 156 having a front surface, and a back surface.

Aim assisting system 100 comprises body 110. Body 110 comprises in functional combination band 114 and eye patch 156. Band 114 comprises cover 118 and eye patch 156. Cover 118 comprises inner surface 122 and outer surface 128. Eye patch 156 comprises front surface and back surface. Eyepatch 156 is integral to band 114, eyepatch 156 affixed parallel to band 114. Back surface of eye patch 156 is adjacent non-aiming-eye 160 of user-wearer 140 during use to facilitate ease of focus by aiming-eye 164 such that vision from non-aiming-eye 160 is blocked while shooting.

Shooting comprises bow and arrow. Shooting comprises firearms. Band 114 comprises headband 170. Band 114 comprises ear warmer 174. Band 114 in elastically deformable. Band 114 is able to be adjusted such that eye patch 156 is able to be moved from non-aiming eye 160 to aiming eye 164 based on user-wearer 140 eye dominance. Insulator 134 is enveloped by outer cover 118. Insulator 134 provided heat transfer resistance. Insulator 134 is enclosed within an inner volume of cover 118.

Eyepatch 156 is able to be moved relative to band 114 as desired. Band 114 comprises a flexible cylindrical profile when used. Band 114 is able to conform to a semi-flat position when folded during a non-use condition. Outer over 118 comprises breathable material. Band 114 is normally in tension. Eye patch 156 is located underneath band 114. Band 114 comprises elastic properties such that band 114 is able to engage with head 142 of user-wearer 140. Aim assisting system 100 may be manufactured and provided for sale in a wide variety of sizes and shapes for a wide assortment of applications.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. An aim assisting system comprising:
a body having;
a band having;
a cover having;
an inner surface; and
an outer surface;
an insulator;
an eye patch having;
a front surface; and
a back surface;
wherein said aim assisting system comprises said body;
wherein said body comprises in functional combination said band and said eye patch;
wherein said band comprises said cover and said eye patch;
wherein said cover comprises said inner surface and said outer surface;
wherein said eye patch comprises said front surface and said back surface wherein said eyepatch is integral to said band, said eyepatch affixed parallel to said band;
wherein said back surface of said eye patch is adjacent a non-aiming-eye of a user-wearer during use to facilitate ease of focus by an aiming-eye such that vision from said non-aiming-eye is blocked while shooting.

2. The aim assisting system of claim 1 wherein said shooting comprises bow and arrow.

3. The aim assisting system of claim 1 wherein said shooting comprises firearms.

4. The aim assisting system of claim 1 wherein said band comprises a headband.

5. The aim assisting system of claim 1 wherein said band comprises an ear warmer.

6. The aim assisting system of claim 1 wherein said band in elastically deformable.

7. The aim assisting system of claim 1 wherein said band is able to be adjusted such that said eye patch is able to be moved from said non-aiming eye to said aiming eye based on said user-wearer eye dominance

8. The aim assisting system of claim 1 wherein said insulator is enveloped by said outer cover.

9. The aim assisting system of claim 9 wherein said insulator provided heat transfer resistance.

10. The aim assisting system of claim 1 wherein said insulator is enclosed within an inner volume of said outer cover.

11. The aim assisting system of claim 1 wherein said eyepatch is able to be moved relative to said band as desired.

12. The aim assisting system of claim 1 wherein said band comprises a flexible cylindrical profile when used.

13. The aim assisting system of claim 1 wherein said band is able to conform to a semi-flat position when folded during a non-use condition.

14. The aim assisting system of claim 1 wherein said outer over comprises breathable material.

15. The aim assisting system of claim 1 wherein said band is normally in tension.

16. The aim assisting system of claim 1 wherein said eye patch is located underneath said band.

17. The aim assisting system of claim 1 wherein said band comprises elastic properties such that said band is able to engage with a head of said user-wearer.

18. An aim assisting system comprising:
a body having;
a band having;
a cover having;
an inner surface; and
an outer surface;
an insulator;
an eye patch having;
a front surface; and
a back surface;
wherein said aim assisting system comprises said body;
wherein said body comprises in functional combination said band and said eye patch;
wherein said band comprises said cover and said eye patch;
wherein said cover comprises said inner surface and said outer surface;
wherein said eye patch comprises said front surface and said back surface
wherein said eyepatch is integral to said band, said eyepatch affixed parallel to said band; and
wherein said back surface of said eye patch is adjacent a non-aiming-eye of a user-wearer during use to facilitate ease of focus by an aiming-eye such that vision from said non-aiming-eye is blocked while shooting.
